(19) United States
(12) Patent Application Publication
(10) Pub. No.: US 2023/0248293 A1
(43) Pub. Date: Aug. 10, 2023

(54) Lead Wire of ECG

(71) Applicants: Guangren Chen, Arcadia, CA (US); Cheng Hong, Shanghai (CN); Zhiqin Yao, Shanghai (CN)

(72) Inventors: Guangren Chen, Arcadia, CA (US); Cheng Hong, Shanghai (CN); Zhiqin Yao, Shanghai (CN)

(21) Appl. No.: 18/166,506

(22) Filed: Feb. 9, 2023

Related U.S. Application Data
(60) Provisional application No. 63/267,770, filed on Feb. 9, 2022.

(51) Int. Cl. A61B 5/30; A61B 5/274; A61B 5/28; A61B 5/308

(52) U.S. Cl. CPC A61B 5/303; A61B 5/274; A61B 5/28; A61B 5/308; A61B 2562/227; A61B 2562/222; A61B 2562/12

(57) ABSTRACT

Apparatus includes a conduit cable, a set of wires, a connector, a set of electrode connectors. Each wire of the set of wires is electrically insulated from every other wire and placed in the conduit cable. The connector is adapted for making physical and electrical contact with an ECG measuring device. The connector is physically connected to the conduit cable. The connector includes a separate electrical contact electrically connected to one end of each wire of the set of wires. Each connector of the set of electrode connectors is adapted for making physical and electrical contact with an ECG electrode. Each connector of the set of electrode connectors is electrically connected to a different wire of the set of wires. Each connector of the set of electrode connectors is physically connected to the conduit cable at a different location along the conduit cable.

### CROSS-REFERENCE TO RELATED APPLICATION

This application claims the benefit of U.S. Provisional Patent Application Ser. No. 63/267,770, filed Feb. 9, 2022, the content of which is incorporated by reference herein in its entirety.

### INTRODUCTION

The teachings herein relate to cables for an electrocardiogram (ECG) measuring device. More particularly, the teachings herein relate to apparatus for transmitting electrical signals from ECG electrodes to an ECG measuring device and methods of manufacturing that apparatus.

### BACKGROUND

An electrocardiogram (ECG) is a measure of the electrical activity of the heart. The electrical signals produced by the heart are picked up by ECG electrodes placed on the surface of the skin. These electrical signals are then conveyed to an ECG measuring device through individual cables connected between each ECG electrode and a connector plugged into the ECG measuring device. These individual cables are sometimes called ECG lead cables.

ECG lead cables or just ECG cables should not be confused with ECG leads. The term “ECG leads” generally refers to measurements made at different angles through the body. Since an ECG lead measurement can be made from one ECG electrode to any other ECG electrode, it is possible to have more ECG lead measurements than the number of ECG cables or ECG electrodes. For example, the standard ECG measuring device produces a 12-lead ECG measurement using ten ECG electrodes and ten ECG cables.

In a standard 12-lead ECG measurement, six ECG electrodes are placed across the chest and four ECG electrodes are attached to the limbs. An ECG cable then electrically connects each ECG electrode to the ECG measuring device. This means that a total of ten cables need to be draped across a patient's body.

Not all ECG measuring devices use ten ECG electrodes. The number varies significantly depending on the type of measurement being done. For example, Holter monitors have as few as three ECG electrodes. Three ECG electrodes, however, still require a patient to deal with three separate electrical cables hanging from their body throughout the entire time their heart activity is being monitored.

Due to the patient's hand or body movement, a cable can easily be detached from an electrode. In addition, the use of multiple cables also means that they can become entangled with each other or snagged by something nearby. Such events prolong or interrupt testing time. As a result, as the number of cables used in ECG measurement increases, so does the risk of poor, interrupted, or delayed results.

Consequently, additional apparatus and methods of manufacturing that apparatus are needed to reduce the number of cables required for ECG measurement.

### BRIEF DESCRIPTION OF THE DRAWINGS

FIG. 1 is an exemplary diagram showing an apparatus that includes two cables for performing a standard 12-lead ECG measurement, in accordance with various embodiments.

FIG. 2 is an exemplary diagram showing how two cables for performing a standard 12-lead ECG measurement are connected to electrodes placed across the body of a patient, in accordance with various embodiments.

FIG. 3 is an exemplary diagram showing an apparatus that includes one cable and five electrode connectors for performing an ECG measurement, in accordance with various embodiments.

FIG. 4 is an exemplary diagram showing an apparatus that includes one cable and three electrode connectors for performing an ECG measurement, in accordance with various embodiments.

FIG. 5 is a flowchart showing a method for manufacturing apparatus for transmitting electrical signals from ECG electrodes adapted for placement on skin to an ECG measuring device, in accordance with various embodiments.

Before one or more embodiments of the invention are described in detail, one skilled in the art will appreciate that the invention is not limited in its application to the details of construction, the arrangements of components, and the arrangement of steps set forth in the following detailed description. The invention is capable of other embodiments and of being practiced or being carried out in various ways. Also, it is to be understood that the phraseology and terminology used herein is for the purpose of description and should not be regarded as limiting.

### DETAILED DESCRIPTION

As described above, the electrical signals produced by the heart are picked up by ECG electrodes placed on the surface of the skin. These electrical signals are then conveyed to an ECG measuring device through individual cables connected between each ECG electrode and a connector plugged into the ECG measuring device.

ECG measuring devices include between three and ten ECG electrodes requiring between three and ten ECG cables to convey their electrical signals. Due to the patient's hand or body movement, a cable can easily be detached from an electrode. In addition, the use of multiple cables also means that they can become entangled with each other or snagged by something nearby. Such events prolong or interrupt testing time. As a result, as the number of cables used in ECG measurement increases, so does the risk of poor measurement results.

Consequently, additional apparatus and methods of manufacturing that apparatus are needed to reduce the number of cables required for ECG measurement.

In various embodiments, ECG measurement is improved by conveying electrical signals picked up by two or more ECG electrodes through a single ECG cable to an ECG measuring device. This single ECG cable includes connectors located along the length of the cable to connect to each of the two or more ECG electrodes. In other words, the wires of traditional ECG cables are placed together in a single conduit cable and the electrode connectors of traditional ECG cables are placed along the conduit cable at different locations. As a result, as little as one cable can be used for some ECG measuring devices.

As described above, in the standard 12-lead ECG measurement, six ECG electrodes are placed across the chest and four ECG electrodes are attached to the limbs. This means that a total of ten cables are needed.

In various embodiments, only two cables are needed for a standard 12-lead ECG measurement. The electrical signals from six ECG electrodes across the chest are conveyed using a first cable. The signals from four ECG electrodes attached to the limbs are conveyed using a second cable.

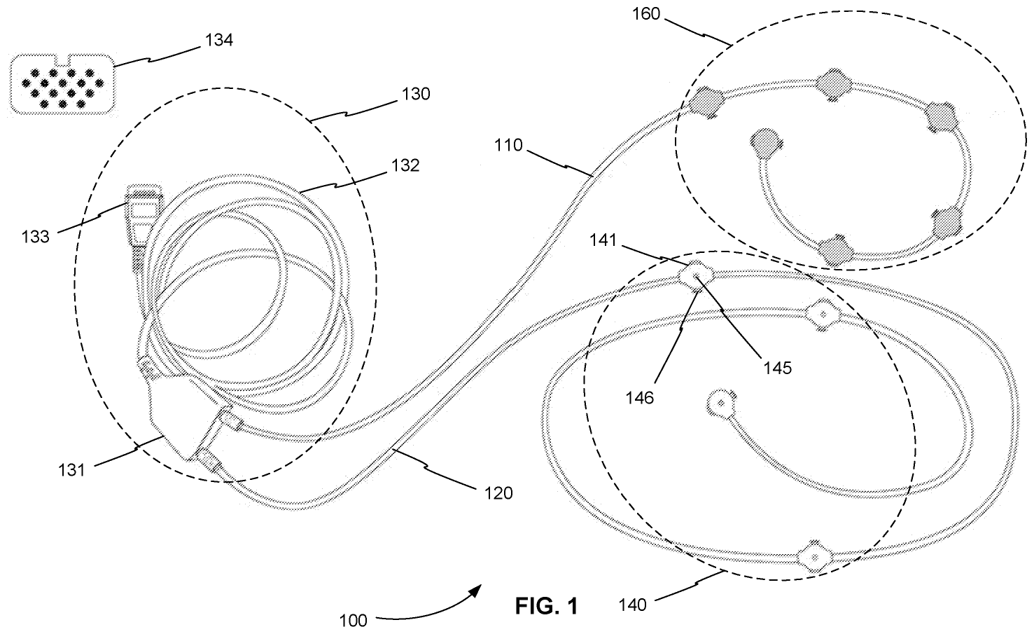

FIG. 1 is an exemplary diagram 100 showing an apparatus that includes two cables for performing a standard 12-lead ECG measurement, in accordance with various embodiments. First conduit cable 110 encloses or includes at least six electrically insulated wires (not shown).

First conduit cable 110 also includes set 160 of six electrode connectors physically connected to first conduit cable 110 at different locations along first conduit cable 110. Each connector of set 160 is adapted for making physical and electrical contact with an ECG electrode adapted for placement on skin. Each connector of set 160 is adapted to connect to the male portion of a button-type ECG electrode, for example. The back of each connector of set 160 is shown in FIG. 1. Each connector of set 160 is also physically and electrically connected to one of the six wires in first conduit cable 110.

Second conduit cable 120 includes at least four electrically insulated wires (not shown). Second conduit cable 120 also includes set 140 of four electrode connectors physically connected to second conduit cable 120 at different locations along second conduit cable 120. Each connector of set 140 is adapted for making physical and electrical contact with an ECG electrode adapted for placement on skin. Each connector of set 140 is also physically and electrically connected to one of the four wires in conduit cable 120.

Each connector of set 140 is adapted to connect to the male portion of a button-type ECG electrode, for example. The front of each connector of set 140 is shown in FIG. 1, for example. Connector 141, for example, includes female portion 145 for connecting with a male portion of a button-type ECG electrode. Connector 141 also includes release mechanism or button 146 that is used to release a male portion of a button-type ECG electrode from female portion 145.

First conduit cable 110 is also physically connected to connector 130. In FIG. 1, connector 130 includes housing 131, cable 132, and interface 133. In various alternative embodiments, connector 130 is one combined unit including an interface and housing.

Connector 130 is adapted to make physical and electrical contact with an ECG measuring device (not shown). Connector 130 makes physical and electrical contact with an ECG measuring device using interface 133, for example.

Connector 130 is also adapted to include a separate electrical contact to electrically connect to each wire of the six electrically insulated wires of first conduit cable 110 and a contact of the ECG measuring device. Separate electrical contacts of interface 133 of connector 130 are shown, for example, in diagram 134. The separate electrical contacts of interface 133 convey the electrical signals to the ECG measuring device.

Similarly, second conduit cable 120 is physically connected to connector 130. Connector 130 is also adapted to include a separate electrical contact to electrically connect to each wire of the four electrically insulated wires of second conduit cable 120 and a contact of the ECG measuring device.

As a result, FIG. 1 shows how the ten cables needed for traditional 12-lead ECG measurement are reduced to two cables.

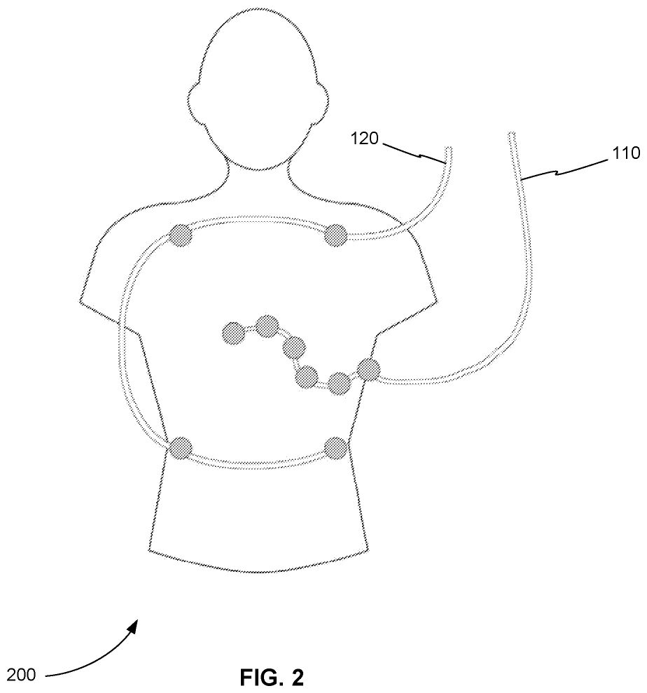

FIG. 2 is an exemplary diagram 200 showing how two cables for performing a standard 12-lead ECG measurement are connected to electrodes placed across the body of a patient, in accordance with various embodiments. The four connectors of conduit cable 120 are connected to limb ECG electrodes, for example. The six connectors of conduit cable 110 are connected to chest ECG electrodes. FIG. 2 shows that reducing the number of cables significantly reduces the clutter of cables across the body of a patient and reduces the risk of cables becoming tangled or snagged. If the locations of the connectors along conduit cables 110 and 120 are fixed, however, tension may be produced on the ECG electrodes if connectors are too far apart or too close together.

As a result, in various embodiments, conduit cables 110 and 120 are further adapted to allow their connectors to be moved to different locations along conduit cables 110 and 120. For example, the wires of conduit cables 110 and 120 can include tracks that the connectors of conduit cables 110 and 120 can physically and electrically connect to. Separate slots in conduit cables 110 and 120 can then allow the connectors to move along their respective tracks and with respect to each other along conduit cables 110 and 120.

A release and stop mechanism similar to the mechanism used to connect to ECG electrodes can be used to release and stop connectors positioned along conduit cables 110 and 120. For example, a button or clamping mechanism that places or releases a stop against the wire track can be used. Allowing the connectors to move along conduit cables 110 and 120 removes any tension between electrodes or any kinking of conduit cables 110 and 120 between electrodes.

FIGS. 1 and 2 show how ten cables can be reduced to two cables. In various alternative embodiments, ten cables can be reduced to one cable. In other words, electrical signals from all ten electrodes can be transmitted to an ECG measuring device using just one cable. Reducing the number of cables to a single cable is especially useful for ECG monitoring devices, including, but not limited to, exercise ECG devices and Holter monitors. Typically, these devices already have fewer electrodes and therefore fewer cables. However, the patient is more likely to move around with these types of ECG measuring devices, making the reduction in the number of cables even more important.

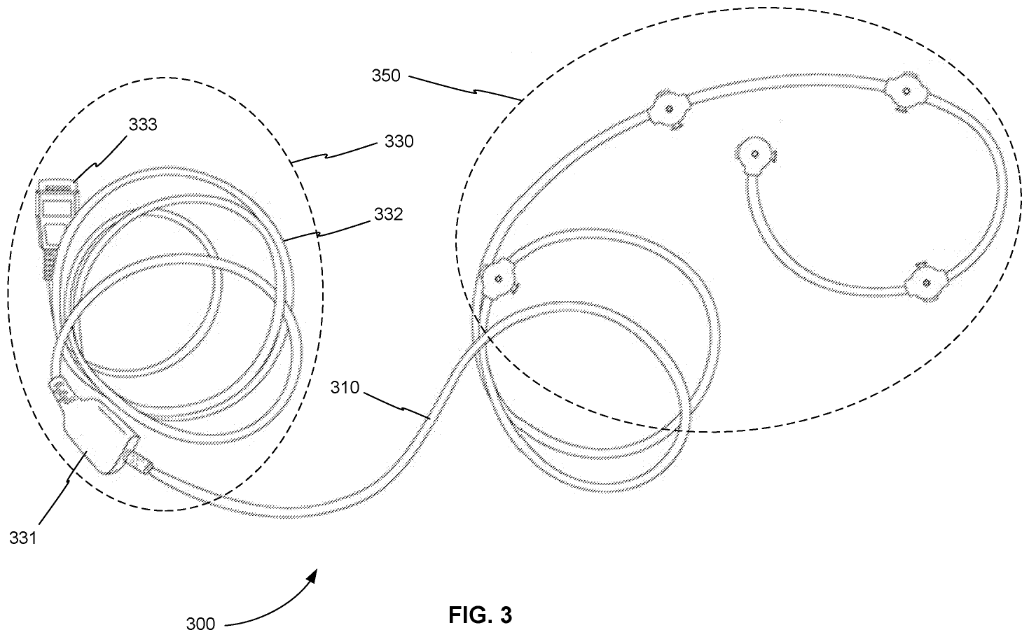

FIG. 3 is an exemplary diagram 300 showing an apparatus that includes one cable and five electrode connectors for performing an ECG measurement, in accordance with various embodiments. Conduit cable 310 encloses or includes at least five electrically insulated wires (not shown).

Conduit cable 310 also includes set 350 of five electrode connectors physically connected to conduit cable 310 at different locations along conduit cable 310. Each connector of set 350 is adapted for making physical and electrical contact with an ECG electrode adapted for placement on skin. Each connector of set 350 is adapted to connect to the male portion of a button-type ECG electrode, for example. The front of each connector of set 350 is shown in FIG. 3. Each connector of set 350 is also physically and electrically connected to one of the five wires in conduit cable 310.

Conduit cable 310 is also physically connected to connector 330. In FIG. 3, connector 330 includes housing 331, cable 332, and interface 333. In various alternative embodiments, connector 330 is one combined unit including an interface and housing.

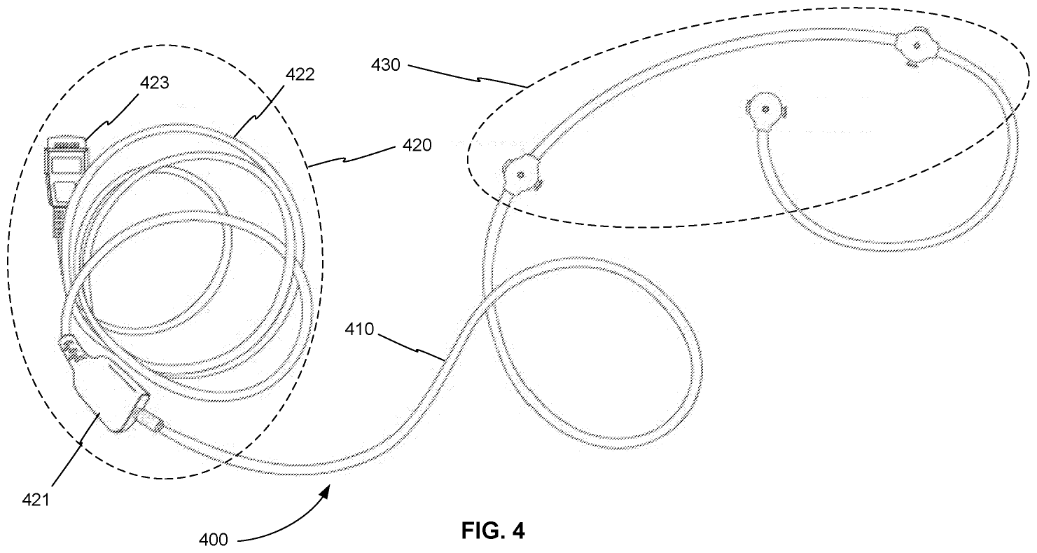

FIG. 4 is exemplary diagram 400 showing an apparatus that includes one cable and three electrode connectors for performing an ECG measurement, in accordance with various embodiments. Conduit cable 410 encloses or includes at least three electrically insulated wires (not shown).

Conduit cable 410 also includes set 430 of three electrode connectors physically connected to conduit cable 410 at different locations along conduit cable 410. Each connector of set 430 is adapted for making physical and electrical contact with an ECG electrode adapted for placement on skin. Each connector of set 430 is adapted to connect to the male portion of a button-type ECG electrode, for example. The front of each connector of set 430 is shown in FIG. 4. Each connector of set 430 is also physically and electrically connected to one of the three wires in conduit cable 410.

Conduit cable 410 is also physically connected to connector 420. In FIG. 4, connector 420 includes housing 421, cable 422, and interface 423. In various alternative embodiments, connector 420 is one combined unit including an interface and housing.

### Apparatus for Transmitting ECG Signals

Returning to FIG. 1, an apparatus is shown for transmitting electrical signals from ECG electrodes adapted for placement on skin to an ECG measuring device. This apparatus includes first conduit cable 110, a first set of two or more wires (not shown), connector 130, and first set 160 of two or more electrode connectors. In the first set of two or more wires, each wire is electrically insulated from every other wire that is placed in first conduit cable 110.

Connector 130 is adapted for making physical and electrical contact with an ECG measuring device. Connector 130 is physically connected to first conduit cable 110. This connection may be a permanent connection or a removeable connection. Connector 130 includes a separate electrical contact electrically connected to one end of each wire of the first set of two or more wires.

Each connector of first set 160 of two or more electrode connectors is adapted for making physical and electrical contact with an ECG electrode adapted for placement on skin. Each connector of first set 160 of two or more electrode connectors is electrically connected to a different wire of the first set of two or more wires. Each connector of first set 160 of two or more electrode connectors is physically connected to first conduit cable 110 at a different location along first conduit cable 110.

In various embodiments, first set 160 of two or more electrode connectors can include three, four, five, six, or ten connectors.

In various embodiments, each connector of first set 160 of two or more electrode connectors is adapted for making physical and electrical contact with a button-type ECG electrode or tab-type ECG electrode.

In various embodiments, each connector of first set 160 of two or more electrode connectors includes a release mechanism for a releasing an ECG electrode adapted for placement on skin. This release mechanism can include, for example, a button mechanism for a button-type ECG electrode or a clamp mechanism for a tab-type ECG electrode.

In various embodiments, first conduit cable 110 is further adapted to allow at least one connector of first set 160 of two or more electrode connectors to be moved to different locations along first conduit cable 110 while still maintaining physical contact with first conduit cable 110 and electrical contact with a wire of the first set of set of two or more wires.

As described above, in one embodiment, the wires of the first set of set of two or more wires are adapted to include tracks that connectors of first set 160 of two or more electrode connectors can physically and electrically connect to. Separate slots in first conduit cable 110 can then allow the connectors to move along their respective tracks and with respect to each other along first conduit cable 110.

In various embodiments, a release and stop mechanism similar to the mechanism used to connect to ECG electrodes can be used to release and stop connectors of first set 160 of two or more electrode connectors positioned along first conduit cable 110. For example, a button or clamping mechanism that places or releases a stop against the wire track can be used. Allowing the connectors of first set 160 of two or more electrode connectors to move along first conduit cable 110 removes any tension between electrodes or any kinking of first conduit cable 110 between electrodes.

In various embodiments, the apparatus further includes second conduit cable 120, second set of two or more wires (not shown), and second set 140 of two or more electrode connectors. In the second set of two or more wires, each wire is electrically insulated from every other wire that is placed in second conduit cable 120. Connector 130 is further is physically connected to second conduit cable 120 and includes a separate electrical contact electrically connected to one end of each wire of the second set of two or more wires.

Each connector of second set 140 of two or more electrode connectors is adapted for making physical and electrical contact with an ECG electrode adapted for placement on skin. Each connector of second set 140 of two or more electrode connectors is electrically connected to a different wire of the second set of two or more wires. Each connector of second set 140 of two or more electrode connectors is physically connected to second conduit cable 120 at a different location along second conduit cable 120.

In various embodiments, second set 140 of two or more electrode connectors can include three, four, five, or six connectors.

In various embodiments, the first set of two or more wires includes six wires and first set 160 of two or more electrode connectors includes six connectors.

In various embodiments, the second set of two or more wires includes four wires and second set 140 of two or more electrode connectors includes four connectors.

In various embodiments, each connector of second set 140 of two or more electrode connectors is adapted for making physical and electrical contact with a button-type ECG electrode or tab-type ECG electrode.

In various embodiments, each connector of second set 140 of two or more electrode connectors includes a release mechanism for a releasing an ECG electrode adapted for placement on skin. This release mechanism can include, for example, a button mechanism for a button-type ECG electrode or a clamp mechanism for a tab-type ECG electrode.

In various embodiments, second conduit cable 120 is further adapted to allow at least one connector of second set 140 of two or more electrode connectors to be moved to different locations along second conduit cable 120 while still maintaining physical contact with second conduit cable 120 and electrical contact with a wire of the second set of set of two or more wires.

As described above, in one embodiment, the wires of second set of set of two or more wires are adapted to include tracks that connectors of second set 140 of two or more electrode connectors can physically and electrically connect to. Separate slots in second conduit cable 120 can then allow the connectors to move along their respective tracks and with respect to each other along second conduit cable 120.

In various embodiments, a release and stop mechanism similar to the mechanism used to connect to ECG electrodes can be used to release and stop connectors of second set 140 of two or more electrode connectors positioned along second conduit cable 120. For example, a button or clamping mechanism that places or releases a stop against the wire track can be used. Allowing the connectors of second set 140 of two or more electrode connectors to move along second conduit cable 120 removes any tension between electrodes or any kinking of second conduit cable 120 between electrodes.

### Method for Manufacturing Apparatus for Transmitting ECG Signals

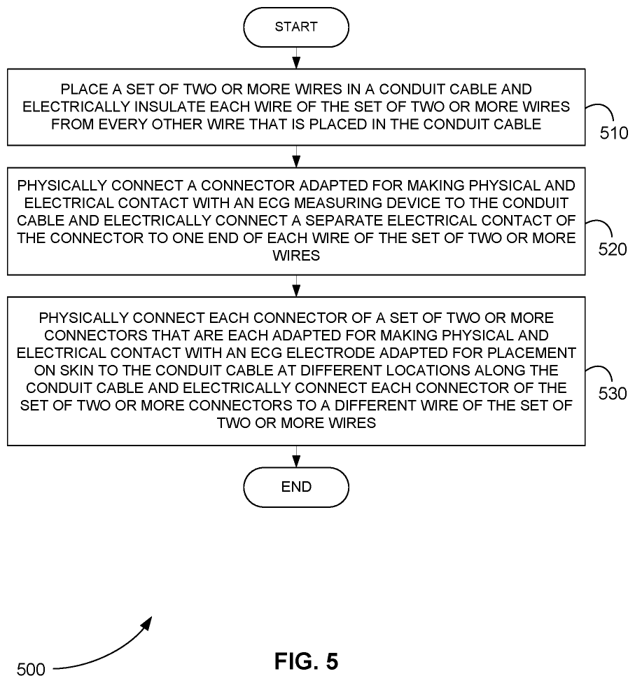

FIG. 5 is a flowchart showing a method 500 for manufacturing apparatus for transmitting electrical signals from ECG electrodes adapted for placement on skin to an ECG measuring device, in accordance with various embodiments.

In step 510 of method 550, a set of two or more wires is placed in a conduit cable and each wire of the set of two or more wires is electrically insulated from every other wire that is placed in the conduit cable.

In step 520, a connector adapted for making physical and electrical contact with an ECG measuring device is physically connected to the conduit cable and a separate electrical contact of the connector is electrically connected to one end of each wire of the set of two or more wires.

In step 530, each connector of a set of two or more connectors that are each adapted for making physical and electrical contact with an ECG electrode adapted for placement on skin is physically connected to the conduit cable at different locations along the conduit cable and each connector of the set of two or more connectors is electrically connected to a different wire of the set of two or more wires.

In various embodiments, the method includes three additional steps. A first step includes placing a second set of two or more wires in a second conduit cable and electrically insulating each wire of the second set of two or more wires from every other wire that is placed in the conduit cable. A second step includes physically connecting the connector adapted for making physical and electrical contact with an ECG measuring device to the conduit cable and electrically connecting a separate electrical contact of the connector to one end of each wire of the second set of two or more wires. A third step includes physically connecting each connector of a second set of two or more connectors that are each adapted for making physical and electrical contact with an ECG electrode adapted for placement on skin to the second conduit cable at different locations along the second conduit cable and electrically connecting each connector of the second set of two or more connectors to a different wire of the second set of two or more wires.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

What is claimed is:

1. Apparatus for transmitting electrical signals from electrocardiogram (ECG) electrodes adapted for placement on skin to an ECG measuring device, comprising:
a conduit cable;
a set of two or more wires with each wire electrically insulated from every other wire that is placed in the conduit cable;
a connector adapted for making physical and electrical contact with an ECG measuring device that is physically connected to the conduit cable and that includes a separate electrical contact electrically connected to one end of each wire of the set of two or more wires;
a set of two or more connectors that are each adapted for making physical and electrical contact with an ECG electrode adapted for placement on skin and that are each electrically connected to a different wire of the set of two or more wires and are physically connected to the conduit cable at different locations along the conduit cable.

2. The apparatus of claim 1, wherein the set of two or more wires comprises three wires and the set of two or more connectors comprises three connectors.

3. The apparatus of claim 1, wherein the set of two or more wires comprises four wires and the set of two or more connectors comprises four connectors.

4. The apparatus of claim 1, wherein the set of two or more wires comprises five wires and the set of two or more connectors comprises five connectors.

5. The apparatus of claim 1, wherein the set of two or more wires comprises six wires and the set of two or more connectors comprises six connectors.

6. The apparatus of claim 1, wherein the set of two or more connectors comprises connecters that are each adapted for making physical and electrical contact with a button-type ECG electrode.

7. The apparatus of claim 1, wherein each connecter of the set of two or more connectors comprises a release mechanism for a releasing an ECG electrode adapted for placement on skin.

8. The apparatus of claim 1, wherein the set of two or more connectors comprises connecters that are each adapted for making physical and electrical contact with a tab-type ECG electrode.

9. The apparatus of claim 1, wherein the conduit cable is further adapted to allow at least one connector of the set of two or more connectors to be moved to different locations along the conduit cable while still maintaining physical contact with the conduit cable and electrical contact with a wire of the set of set of two or more wires.

10. The apparatus of claim 9, wherein the at least one connector comprises a stop and release mechanism for stopping the at least one connector from moving to different locations along the conduit cable and releasing the at least one connector to enable movement to different locations along the conduit cable.

11. The apparatus of claim 1, further comprising
a second conduit cable;
a second set of two or more wires with each wire electrically insulated from every other wire that is placed in the second conduit cable, wherein the connector is further is physically connected to the second conduit cable and includes a separate electrical contact electrically connected to one end of each wire of the second set of two or more wires; and
a second set of two or more connectors that are each adapted for making physical and electrical contact with an ECG electrode adapted for placement on skin and that are each electrically connected to a different wire of the second set of two or more wires and are physically connected to the second conduit cable at different locations along the second conduit cable.

12. The apparatus of claim 11, wherein the set of two or more wires comprises six wires and the set of two or more connectors comprises six connectors.

13. The apparatus of claim 11, wherein the second set of two or more wires comprises four wires and the second set of two or more connectors comprises four connectors.

14. The apparatus of claim 11, wherein the second set of two or more connectors comprises connecters that are each adapted for making physical and electrical contact with a button-type ECG electrode.

15. The apparatus of claim 11, wherein each connecter of the second set of two or more connectors comprises a release mechanism for a releasing an ECG electrode adapted for placement on skin.

16. The apparatus of claim 11, wherein the second set of two or more connectors comprises connecters that are each adapted for making physical and electrical contact with a tab-type ECG electrode.

17. The apparatus of claim 11, wherein the second conduit cable is further adapted to allow at least one connector of the second set of two or more connectors to be moved to different locations along the second conduit cable while still maintaining physical contact with the second conduit cable and electrical contact with a wire of the second set of set of two or more wires.

18. The apparatus of claim 17, wherein the at least one connector of the second set comprises a stop and release mechanism for stopping the at least one connector from moving to different locations along the second conduit cable and releasing the at least one connector of the second set to enable movement to different locations along the second conduit cable.

19. A method for manufacturing apparatus for transmitting electrical signals from electrocardiogram (ECG) electrodes adapted for placement on skin to an ECG measuring device, comprising:
placing a set of two or more wires in a conduit cable and electrically insulating each wire of the set of two or more wires from every other wire that is placed in the conduit cable;
physically connecting a connector adapted for making physical and electrical contact with an ECG measuring device to the conduit cable and electrically connecting a separate electrical contact of the connector to one end of each wire of the set of two or more wires; and
physically connecting each connector of a set of two or more connectors that are each adapted for making physical and electrical contact with an ECG electrode adapted for placement on skin to the conduit cable at different locations along the conduit cable and electrically connecting each connector of the set of two or more connectors to a different wire of the set of two or more wires.

20. The method of claim 19, further comprising:
placing a second set of two or more wires in a second conduit cable and electrically insulating each wire of the second set of two or more wires from every other wire that is placed in the conduit cable;
physically connecting the connector adapted for making physical and electrical contact with an ECG measuring device to the conduit cable and electrically connecting a separate electrical contact of the connector to one end of each wire of the second set of two or more wires; and
physically connecting each connector of a second set of two or more connectors that are each adapted for making physical and electrical contact with an ECG electrode adapted for placement on skin to the second conduit cable at different locations along the second conduit cable and electrically connecting each connector of the second set of two or more connectors to a different wire of the second set of two or more wires.
